(19) United States
(12) Patent Application Publication
(10) Pub. No.: US 2025/0297231 A1
(43) Pub. Date: Sep. 25, 2025

(54) CELL DETACHMENT METHOD, CELL DETACHMENT APPARATUS, AND CELL CRYOPRESERVATION SYSTEM

(71) Applicants: Canon Medical Systems Corporation, Otawara-shi (JP); CANON KABUSHIKI KAISHA, Tokyo (JP)

(72) Inventors: Yuhi HIGUCHI, Tokyo (JP); Shuhei IKEDA, Yokohama (JP); Yusuke OWAKI, Moka (JP)

(73) Assignees: Canon Medical Systems Corporation, Otawara-shi (JP); CANON KABUSHIKI KAISHA, Tokyo (JP)

(21) Appl. No.: 19/081,353

(22) Filed: Mar. 17, 2025

(30) Foreign Application Priority Data
Mar. 19, 2024 (JP) 2024-043771

(51) Int. Cl. C12N 5/00; C12M 1/00

(52) U.S. Cl. CPC C12N 5/562; C12M 29/00; C12M 47/02; C12N 5/525

(57) ABSTRACT

According to one embodiment, a cell detachment method comprising: a detachment step of discharging a liquid used for cryopreservation of cells toward a surface of a culture container in contact with the cells, where the cells are cultured, thereby detaching the cells from the surface; and a recovery step of recovering the detached cells together with the discharged liquid used for cryopreservation.

### CROSS-REFERENCE TO RELATED APPLICATIONS

This application is based upon and claims the benefit of priority from Japanese Patent Application No. 2024-043771, filed Mar. 19, 2024, the entire contents of which are incorporated herein by reference.

### FIELD

Embodiments described herein relate generally to a cell detachment method, a cell detachment apparatus, and a cell cryopreservation system.

### BACKGROUND

After cells are cultured in a culture container, the cells are detached from the culture container using a saline solution or a detachment liquid. When preserving the detached cells for a long time without passage, the solvent of a suspension in which the detached cells are suspended (to be referred to as a cell-suspended detachment liquid hereinafter) is replaced with a cryopreservation liquid, and the cryopreservation liquid in which the cells are suspended (to be referred to as a cell-suspended preservation liquid hereinafter) is frozen and preserved.

One of liquid replacement methods is a method using a filter (to be referred to as a filter liquid replacement method hereinafter). In the filter liquid replacement method, cells are captured by passing a cell-suspended detachment liquid through a filter, and the cells are recovered by passing a cryopreservation liquid through the filter. In the filter liquid replacement method, however, many of the cells suspended in the cell-suspended detachment liquid cannot be recovered, and loss of cells is large.

### BRIEF DESCRIPTION OF THE DRAWINGS

FIG. 1 is a view showing an example of the configuration of a cell cryopreservation system.

FIG. 2 is a view showing an example of the configuration of a cell detachment apparatus.

FIG. 3 is a view schematically showing the outer appearance of a detachment mechanism.

FIG. 4 is a flowchart showing an example of the processing procedure of detachment/cryopreservation by the cell cryopreservation system.

FIG. 5 is a view schematically showing the processing procedure of steps S1 to S8 in FIG. 4.

FIG. 6 is a view schematically showing the processing procedure of a recovery step (S8).

FIG. 7 is a view showing comparison of the number of iPS cells after thawing and culture between the embodiment and a comparison example.

### DETAILED DESCRIPTION

A cell detachment method according to the embodiment includes a detachment step and a recovery step. In the detachment step, a liquid used for cryopreservation of cells is discharged toward a surface of a culture container in contact with the cells, where the cells are cultured, thereby detaching the cells from the surface. In the recovery step, the detached cells are recovered together with the discharged liquid used for cryopreservation.

A cell detachment method, a cell detachment apparatus, and a cell cryopreservation system according to this embodiment will now be described in detail with reference to the accompanying drawings.

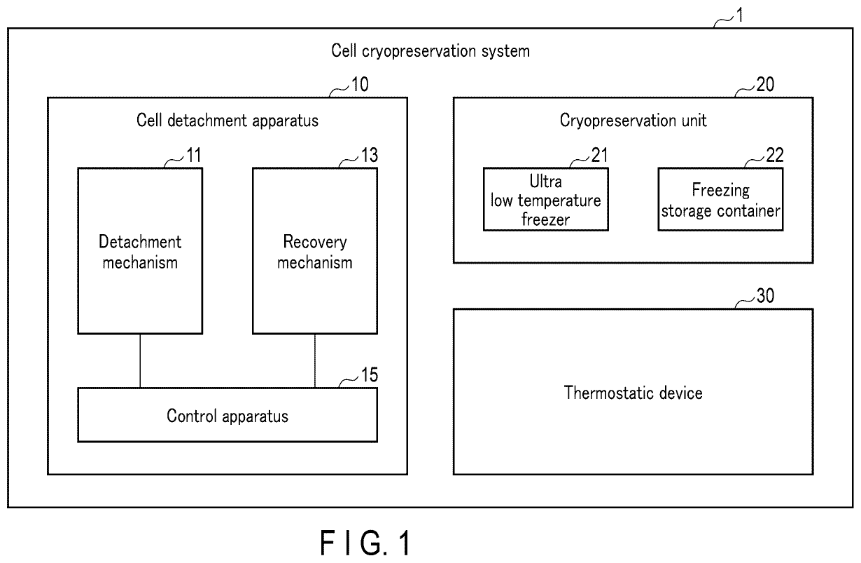

FIG. 1 is a view showing an example of the configuration of a cell cryopreservation system 1 according to this embodiment. As shown in FIG. 1, the cell cryopreservation system 1 is a mechanical system that freezes and preserves cells. The cell according to this embodiment is not particularly limited, and any type of cell can be used. Examples are an iPS (induced Pluripotent Stem) cell, an epithelial cell, an endothelial cell, a synovial cell, a myocardial cell, a myoblastic cell, a fibroblastic cell, and a neuroblastic cell.

As shown in FIG. 1, the cell cryopreservation system 1 includes a cell detachment apparatus 10, a cryopreservation unit 20, and a thermostatic device (incubator) 30. The cell detachment apparatus 10 is a mechanical apparatus that detaches cells cultured in a culture container from the culture container and recovers the detached cells from a culture container. More specifically, the cell detachment apparatus 10 includes a detachment mechanism 11, a recovery mechanism 13, and a control apparatus 15. The detachment mechanism 11 is a mechanical mechanism including a tool configured to detach the cells cultured in the culture container from the culture container. The detachment mechanism 11 discharges a liquid (to be referred to as a cryopreservation liquid hereinafter) used for cryopreservation of cells toward a surface (to be referred to as a culture surface hereinafter) of the culture container in contact with the cells, where the cells are cultured, thereby detaching the cells from the culture surface. The recovery mechanism 13 is a mechanical mechanism including a tool configured to recover, from the culture container, the cells detached from the culture container. The recovery mechanism 13 recovers the cells detached by the detachment mechanism 11 together with the discharged cryopreservation liquid. The cryopreservation liquid in which the cells are suspended will be referred to as a cell-suspended preservation liquid. The control apparatus 15 is a computer including a processor that generally controls the recovery mechanism 13 and the detachment mechanism 11. The recovery mechanism 13 and the detachment mechanism 11 operate in accordance with an instruction from the control apparatus 15.

The cryopreservation unit 20 is a mechanical system that freezes the cells recovered by the cell detachment apparatus 10 and then preserves these. More specifically, the cryopreservation unit 20 includes an ultra low temperature freezer 21 and a freezing storage container 22. The ultra low temperature freezer 21 freezes the cell-suspended preservation liquid in a first temperature range in which the cells can be metabolized. The ultra low temperature freezer 21 includes a heat insulating freezer, and a cooling/circulation device that performs circulation of a refrigerant into the freezer and cooling of the refrigerant. As one example, the first temperature range is assumed to be about −90° C. to −50° C. The ultra low temperature freezer 21 is also called a deep freezer. After a first freezing step by the ultra low temperature freezer 21, the freezing storage container 22 freezes and preserves the cell-suspended preservation liquid in a second temperature range lower than the first temperature range, in which the cells cannot be metabolized. In this case, the second temperature range is assumed to be −200° C. to −150° C.

The thermostatic device 30 includes a chamber in which the culture container is stored, and a control circuit that keeps the temperature and/or humidity in the chamber constant. The thermostatic device 30 may be formed separately from the cell detachment apparatus 10, or the cell detachment apparatus 10 may include the thermostatic device 30. In the latter case, as one example, some or all of the constituent elements of the detachment mechanism 11 and the recovery mechanism 13 are preferably provided in the chamber. With this configuration, cells can be cultured by the cell detachment apparatus 10.

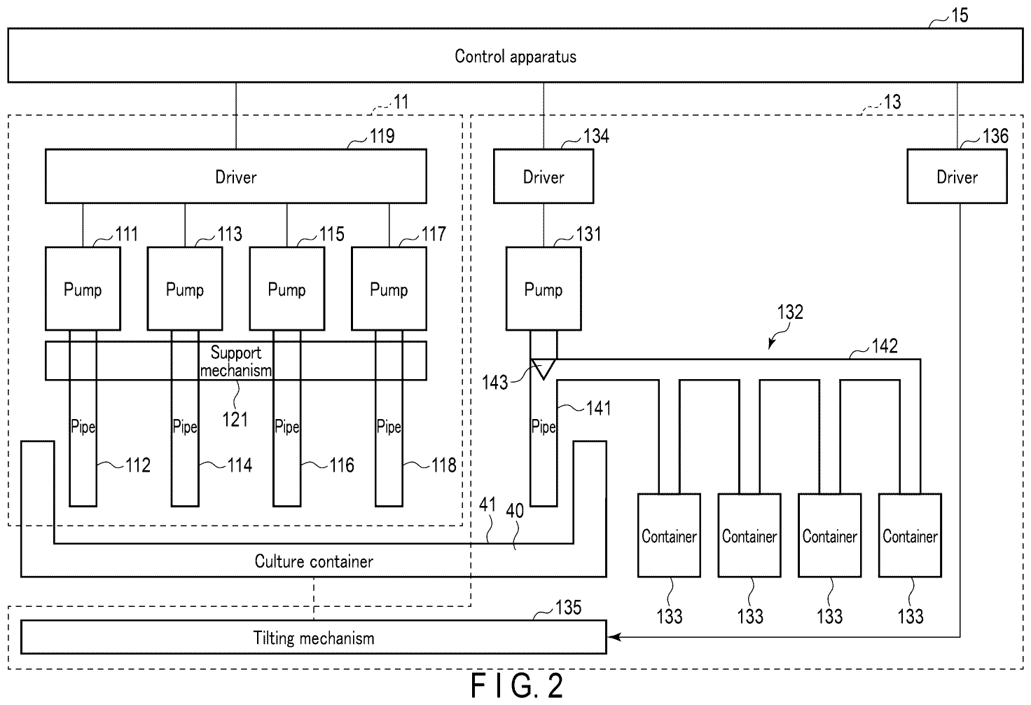

FIG. 2 is a view showing an example of the configuration of the cell detachment apparatus 10. As shown in FIG. 2, the cell detachment apparatus 10 includes the detachment mechanism 11, the recovery mechanism 13, and the control apparatus 15. The detachment mechanism 11, the recovery mechanism 13, and the control apparatus 15 are connected via wired or wireless signal channels. The detachment mechanism 11 includes a detachment liquid pump 111, a liquid feed pipe 112, a cleaning liquid pump 113, a liquid feed pipe 114, a cryopreservation liquid pump 115, a liquid feed pipe 116, a waste liquid pump 117, a suction pipe 118, a driving device 119, and a support mechanism 121.

As shown in FIG. 2, a culture container 40 is installed at a position where the detachment mechanism 11 and the recovery mechanism 13 can access it. Cells are cultured in the culture container 40. The shape of the culture container 40 is not particularly limited, and a dish, a petri dish, a flask, or a well plate having an opening through which the detachment mechanism 11 and the recovery mechanism 13 can access the inside of the culture container 40 is assumed to be used. The inner bottom surface of the culture container 40 is called a culture surface 41. Cultured cells are in contact with the culture surface 41. Before cell detachment processing by the detachment mechanism 11, a plurality of clusters (colonies) of cells are formed in the culture container 40 and adhere to the culture surface 41.

The detachment liquid pump 111 discharges a detachment liquid via the liquid feed pipe 112 in response to a driving signal from the driving device 119. The detachment liquid has an effect of weakening adhesion between the cells and the culture surface 41 or adhesion between the cells. As one example, the detachment liquid is a solution containing a proteolytic enzyme and/or a chelating agent. The detachment liquid is stored in a detachment liquid tank (not shown). The detachment liquid pump 111 sucks the detachment liquid from the detachment liquid tank, and discharges the sucked detachment liquid to the culture container 40 via the liquid feed pipe 112, thereby adding the detachment liquid to the cells included in the culture container 40.

The liquid feed pipe 112 is a tubular structure which is connected to the detachment liquid pump 111 and in which the detachment liquid is distributed, and is, for example, a tube. The liquid feed pipe 112 is supported by the support mechanism 121 such that it can vertically move. The liquid feed pipe 112 is moved downward by the support mechanism 121 toward the culture surface 41 to discharge the detachment liquid, and rises after the end of discharge of the detachment liquid. The support mechanism 121 may be in a form incapable of vertical movement.

The cleaning liquid pump 113 discharges a cleaning liquid via the liquid feed pipe 114 in response to a driving signal from the driving device 119. The cleaning liquid is used to wash out the culture container 40 or impurities. As one example, a saline solution or a liquid culture medium is used as the cleaning liquid. The cleaning liquid is stored in a cleaning liquid tank (not shown). The cleaning liquid pump 113 sucks the cleaning liquid from the cleaning liquid tank, and discharges the sucked cleaning liquid to the culture container 40 via the liquid feed pipe 114, thereby cleaning the culture container 40 and/or the cells. The impurities are, for example, dead cells floating in the cleaning liquid, or calcium ions or magnesium ions existing in the culture medium in the culture container 40. If cleaning is insufficient, the chelating agent may not sufficiently cut bonds between cells. If cleaning is insufficient, calcium ions or magnesium ions existing in the culture medium may react with the chelating agent.

The liquid feed pipe 114 is a tubular structure which is connected to the cleaning liquid pump 113 and in which the cleaning liquid is distributed, and is, for example, a tube. The liquid feed pipe 114 is supported by the support mechanism 121 such that it can vertically move. The liquid feed pipe 114 is moved downward by the support mechanism 121 toward the culture surface 41 to discharge the cleaning liquid, and rises after the end of discharge of the cleaning liquid.

The cryopreservation liquid pump 115 discharges a cryopreservation liquid via the liquid feed pipe 116 in response to a driving signal from the driving device 119. The cryopreservation liquid is used to reduce damages to cells when freezing and preserving the cells. The cryopreservation liquid is a solution containing a cryoprotectant agent. The cryoprotectant agent has an effect of promoting dehydration of cells, lowering the crystallization speed of ice, and impeding formation of ice. This effect suppresses damages to the membranes of cells by ice crystals. As one example, the cryoprotectant agent contains dimethyl sulfoxide (DMSO), glycerol, polyethylene glycol, propylene glycol, glycerin, polyvinyl pyrrolidone, sorbitol, dextran and/or trehalose. As the cryopreservation liquid, a commercially available product such as STEM-CELLBANKER® may be used, or a liquid obtained by mixing a cryoprotectant agent in a culture medium may be used. The cryopreservation liquid is stored in a cryopreservation liquid tank (not shown). The cryopreservation liquid pump 115 sucks the cryopreservation liquid from the cryopreservation liquid tank, and discharges the sucked cryopreservation liquid to the culture surface 41 of the culture container 40 via the liquid feed pipe 116, thereby detaching, from the culture surface 41, the cells in contact with the culture surface 41.

The liquid feed pipe 116 is a tubular structure which is connected to the cryopreservation liquid pump 115 and in which the cryopreservation liquid pump is distributed, and is, for example, a tube. The liquid feed pipe 116 is supported by the support mechanism 121 such that it can vertically move. At the time of discharge of the cryopreservation liquid, the distal end portion of the liquid feed pipe 116 is moved downward by the support mechanism 121 toward the culture surface 41, and rises and moves away from the culture surface 41 after the end of discharge of the cryopreservation liquid.

The waste liquid pump 117 sucks various kinds of waste liquids included in the culture container 40 via the suction pipe 118 in response to a driving signal from the driving device 119. The waste liquid is assumed to be, for example, the detachment liquid or the cleaning liquid discharged to the culture container 40. The sucked waste liquid is stored in a waste liquid tank via a waste tube (not shown).

The suction pipe 118 is a tubular structure which is connected to the waste liquid pump 117 and in which the waste liquid is distributed, and is, for example, a tube. The suction pipe 118 is supported by the support mechanism 121 such that it can vertically move. The suction pipe 118 is moved downward by the support mechanism 121 toward the culture surface 41 to suck the waste liquid, and rises after the end of suction of the waste liquid.

The driving device 119 individually supplies driving signals to the detachment liquid pump 111, the cleaning liquid pump 113, the cryopreservation liquid pump 115, and the waste liquid pump 117 in accordance with an instruction from the control apparatus 15, thereby operating the detachment liquid pump 111, the cleaning liquid pump 113, the cryopreservation liquid pump 115, and the waste liquid pump 117 in accordance with the driving signals. Also, the driving device 119 supplies a driving signal to the support mechanism 121 in accordance with an instruction from the control apparatus 15, thereby vertically moving the liquid feed pipe 112, the liquid feed pipe 114, the liquid feed pipe 116, or the suction pipe 118 in accordance with the driving signal. As one example, a motor is used as the driving device 119.

Note that in FIG. 2, the driving device 119 drives all the detachment liquid pump 111, the cleaning liquid pump 113, the cryopreservation liquid pump 115, the waste liquid pump 117, and the support mechanism 121 solely. However, the embodiment is not limited to this. The driving device 119 may be provided for each of the detachment liquid pump 111, the cleaning liquid pump 113, the cryopreservation liquid pump 115, the waste liquid pump 117, and the support mechanism 121, or the driving device 119 may be provided for each arbitrary combination of the detachment liquid pump 111, the cleaning liquid pump 113, the cryopreservation liquid pump 115, the waste liquid pump 117, and the support mechanism 121.

The recovery mechanism 13 includes a recovery pump 131, a suction pipe 132, a cryopreservation container 133, a driving device 134, a tilting mechanism 135, and a driving device 136.

The recovery pump 131 recovers, via the suction pipe 132, the cells detached by the discharge of the cryopreservation liquid together with the cryopreservation liquid in response to a driving signal from the driving device 134. As described above, the cryopreservation liquid in which the cells are suspended will be referred to as a cell-suspended preservation liquid. The cell-suspended preservation liquid is stored in the cryopreservation container 133.

More specifically, the suction pipe 132 is a tubular structure which is connected to the recovery pump 131 and in which the cell-suspended preservation liquid is distributed. The suction pipe 132 includes a suction branch pipe 141 configured to suck the cell-suspended preservation liquid from the culture container 40, a liquid feed branch pipe 142 configured to feed the sucked cell-suspended preservation liquid to the cryopreservation container 133, and a valve 143 configured to feed the sucked cell-suspended preservation liquid from the suction branch pipe 141 to the liquid feed branch pipe 142. The valve 143 is switched in response to a driving signal from the driving device 134.

The suction branch pipe 141 is supported by the support mechanism 121 such that it can vertically move. At the time of suction of the cell-suspended preservation liquid, the distal end portion of the suction branch pipe 141 is moved downward by the support mechanism 121 toward the culture surface 41, and rises and moves away from the culture surface 41 after the end of suction of the cell-suspended preservation liquid.

A plurality of cryopreservation containers 133 are detachably connected to the liquid feed branch pipe 142. The cell-suspended preservation liquid sucked by the suction branch pipe 141 is distributed through the liquid feed branch pipe 142 and sequentially stored in the plurality of cryopreservation containers 133. In FIG. 2, as one example, four cryopreservation containers 133 are connected. The cryopreservation container 133 has a material and/or structure having such a degree of strength that it does not break even in cryopreservation in the second temperature range by the freezing storage container 22. As one example, a cryotube made of polypropylene, a froze-bag made of a polyolefin-based material, or the like is preferably used as the cryopreservation container 133. Note that as for the number of cryopreservation containers 133 connected to the liquid feed branch pipe 142, a plurality of cryopreservation containers 133 need not always be connected, and one cryopreservation container 133 may be connected.

The driving device 134 supplies a driving signal to the recovery pump 131 in accordance with an instruction from the control apparatus 15, thereby operating the recovery pump 131 in accordance with the driving signal. As one example, a motor is used as the driving device 134.

The tilting mechanism 135 is a mechanical mechanism configured to tilt the culture container 40 with respect to the horizontal direction in response to a driving signal from the driving device 136. More specifically, the tilting mechanism 135 tilts the culture container 40 with respect to the horizontal direction such that the depth of the cell-suspended preservation liquid at a first position where the suction pipe 132 is inserted into the culture container 40 becomes deeper than the depth of the cell-suspended preservation liquid at a second position. The detailed structure of the tilting mechanism 135 is not particularly limited.

The driving device 136 supplies a driving signal to the tilting mechanism 135 in accordance with an instruction from the control apparatus 15, thereby operating the tilting mechanism 135 in accordance with the driving signal. As one example, a motor is used as the driving device 136.

The control apparatus 15 controls the driving device 119, the driving device 134, and the driving device 136 in accordance with a predetermined order to automatically perform detachment and recovery of cells via the detachment liquid pump 111, the cleaning liquid pump 113, the cryopreservation liquid pump 115, the waste liquid pump 117, the recovery pump 131, and the tilting mechanism 135. At this time, the control apparatus 15 controls the flow rate of discharge and suction of the detachment liquid, the cleaning liquid, the cryopreservation liquid, and the cell-suspended preservation liquid, and controls the timings of discharge and suction of the various kinds of liquids described above.

In the following explanation of the embodiment, a “cell” is assumed to be an iPS cell. The detachment mechanism 11 sprays the cryopreservation liquid to the culture surface 41, thereby detaching iPS cells from the culture surface 41.

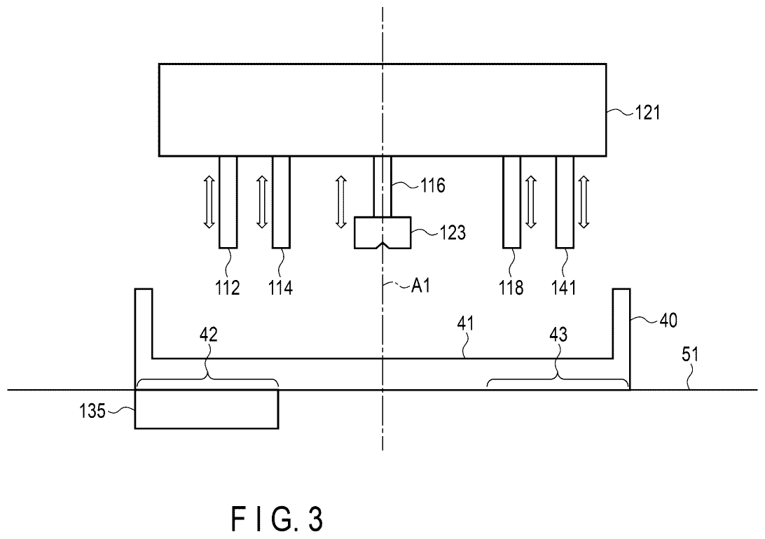

FIG. 3 is a view schematically showing the outer appearance of the detachment mechanism 11. As shown in FIG. 3, the detachment mechanism 11 includes the support mechanism 121. The support mechanism 121 is a structure that supports the liquid feed pipe 112, the liquid feed pipe 114, the liquid feed pipe 116, the suction pipe 118, and the suction branch pipe 141 such that these can individually vertically move. As one example, a ball screw or a linear guide is used as the support mechanism 121.

A placement surface 51 is provided under the liquid feed pipe 112, the liquid feed pipe 114, the liquid feed pipe 116, the suction pipe 118, and the suction branch pipe 141, and the culture container 40 is installed on a portion of the placement surface 51 immediately under the liquid feed pipe 114, the liquid feed pipe 116, the suction pipe 118, and the suction branch pipe 141.

A nozzle 123 is provided at the distal end portion of the liquid feed pipe 116. The nozzle 123 is a mechanical component configured to discharge the cryopreservation liquid fed by the cryopreservation liquid pump 115 via the liquid feed pipe 116 as a plurality of droplets. More specifically, the nozzle 123 sprays the cryopreservation liquid, that is, converts the cryopreservation liquid into a plurality of fine droplets and discharges a mist. To convert the cryopreservation liquid into fine droplets in a mist form, fine holes are formed in the nozzle 123. The number of holes may be one or more. The cryopreservation liquid fed by the cryopreservation liquid pump 115 to the nozzle 123 via the liquid feed pipe 116 is passed through the fine holes formed in the nozzle 123, thus formed into fine droplets, and discharged. Thus, the cryopreservation liquid is sprayed to the culture surface 41 of the culture container 40, and the cells in contact with the culture surface 41 are detached by the dynamic action of the sprayed cryopreservation liquid. Note that the diameter, the discharge speed, the liquid amount, the range, and the like of the droplets of the cryopreservation liquid can arbitrarily be set.

To discharge the cryopreservation liquid to the entire culture surface 41, the liquid feed pipe 116 and the nozzle 123 are preferably mounted on the support mechanism 121 such that these are located substantially above an approximate center A1 of the culture surface 41. Note that the liquid feed pipe 116 and the nozzle 123 may integrally be formed.

The tilting mechanism 135 is buried under the placement surface 51. The tilting mechanism 135 is assumed to be a balloon that can be expanded and contracted by putting air in/out. In this case, the driving device 136 is assumed to be an air compressor, a motor cylinder, or an air cylinder, which puts air into/from the balloon 135. The balloon 135 is installed under one end portion 42 (to be referred to as a rising-side end portion hereinafter) of the culture container 40. In a case where an air compressor is used, the air compressor puts air into the balloon 135 and thus expands it, and the balloon 135 pushes the rising-side end portion 42 upward, thereby tilting the culture container 40. Thus, the waste liquid or cell-suspended preservation liquid included in the culture container 40 gathers to the other end portion (to be referred to as a fixed-side end portion hereinafter) 43 of the culture container 40. The fixed-side end portion 43 indicates a region that exists on the opposite side of the rising-side end portion 42 across the approximate center A1 of the culture surface 41.

To facilitate suction of a solution from the culture container 40, the suction pipe 118 and the suction branch pipe 141 are preferably mounted on the support mechanism 121 such that these are located above the fixed-side end portion 43. The positions of the liquid feed pipe 112 and the liquid feed pipe 114 are not particularly limited, and these are preferably provided at such positions that do not impede the operations of the liquid feed pipe 116, the suction pipe 118, and the suction branch pipe 141.

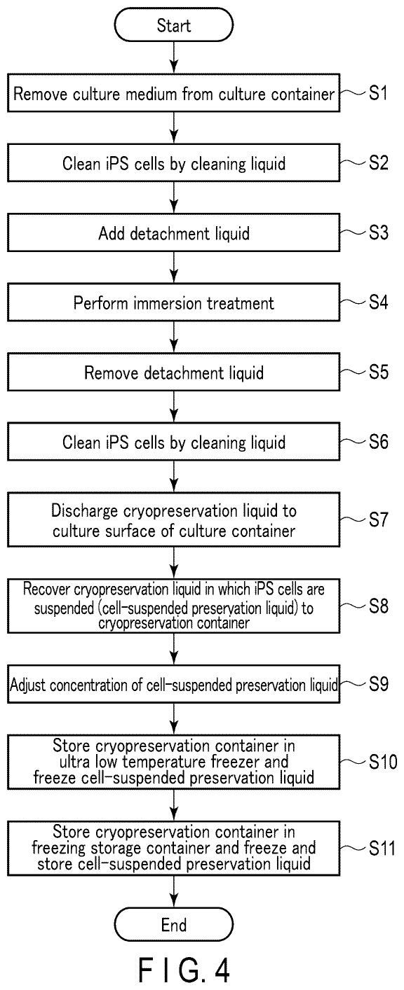

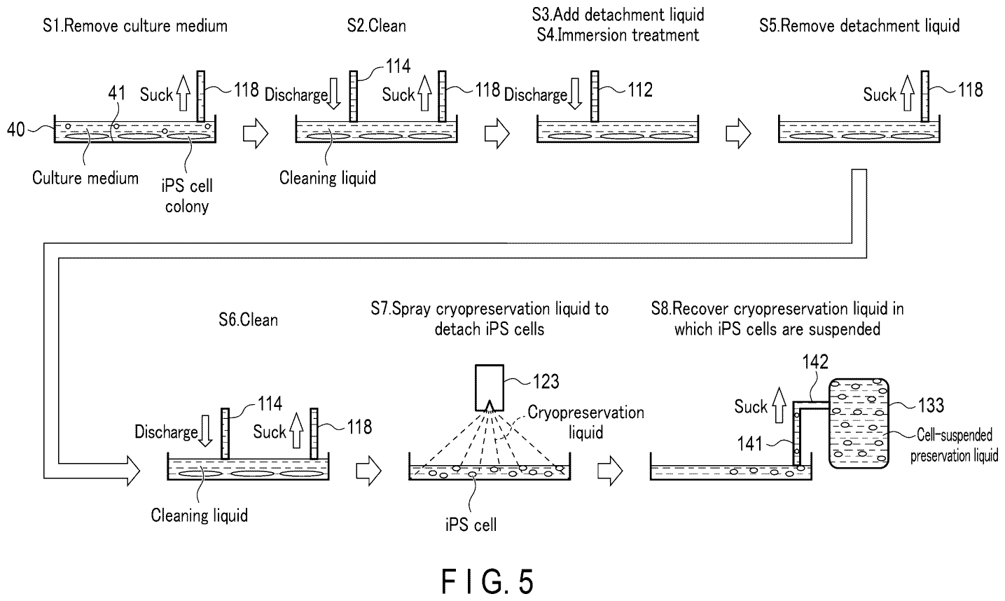

The processing procedure of detachment/cryopreservation by the cell cryopreservation system 1 will be described next with reference to FIGS. 4 and 5. FIG. 4 is a flowchart showing an example of the processing procedure of detachment/cryopreservation by the cell cryopreservation system 1. Steps S1 to S8 shown in FIG. 4 are automatically performed by sequence control and/or feedback control for the driving device 119 of the detachment mechanism 11 and the driving device 134 and the driving device 136 of the recovery mechanism 13 by the control apparatus 15. FIG. 5 is a view schematically showing the processing procedure of steps S1 to S8 in FIG. 4. FIG. 5 shows only components necessary for the explanation of each step, and components that are not necessary for the explanation are appropriately omitted. Note that the thermostatic device 30 and the cell detachment apparatus 10 are assumed to be integrally formed. More specifically, the liquid feed pipe 112, the liquid feed pipe 114, the liquid feed pipe 116, the suction pipe 118, and the nozzle 123 are provided in the chamber of the thermostatic device 30.

As shown in FIGS. 4 and 5, at the start of step S1, the culture container 40 is placed on the placement surface 51. Colonies of cultured iPS cells and a culture medium used for the culture are stored in the culture container 40. The culture of the iPS cells may be performed by the thermostatic device 30 or may be performed by another thermostatic device.

First, the culture medium is removed from the culture container 40 (step S1). More specifically, the control apparatus 15 operates the waste liquid pump 117 via the driving device 119. The waste liquid pump 117 sucks the culture medium from the culture container 40 via the suction pipe 118, thereby removing the culture medium from the culture container 40. Impurities such as the culture medium remaining without being sucked remain in the culture container 40.

If step S1 is performed, the iPS cells are cleaned by the cleaning liquid (step S2). In the culture medium, impurities including nutritional components are included, in addition to the iPS cells. In step S2, the control apparatus 15 operates the cleaning liquid pump 113 via the driving device 119. The cleaning liquid pump 113 discharges the cleaning liquid into the culture container 40 via the liquid feed pipe 114. The impurities are washed out by the discharged cleaning liquid. After that, the waste liquid pump 117 sucks the cleaning liquid containing the washed-out impurities from the culture container 40 via the suction pipe 118. The culture container 40 or the iPS cells are thus cleaned.

If step S2 is performed, the detachment liquid is added (step S3). In step S3, the control apparatus 15 operates the detachment liquid pump 111 via the driving device 119. The detachment liquid pump 111 discharges the detachment liquid into the culture container 40 via the liquid feed pipe 112. The discharged detachment liquid is added to the iPS cells.

If step S3 is performed, an immersion treatment is performed (step S4). The immersion treatment can include incubating. If incubating is included, more specifically, the control apparatus 15 notifies the thermostatic device 30 of an incubating start instruction. Upon receiving the start instruction, the thermostatic device 30 keeps the environment of the culture container 40 including the iPS cells with the detachment liquid added thereto at a constant temperature and humidity for a predetermined thermostatic period. The constant temperature and humidity are preferably set to conditions suitable for culture of iPS cells. For example, it is preferable that the temperature is set to about 37° C., and the humidity is set to about 95%. During incubating, adhesion between the iPS cells and adhesion between the iPS cells and the culture surface 41 are reduced by the effect of the detachment liquid. The thermostatic period is preferably set to a value empirically determined as a time length for sufficiently reducing adhesion. After the elapse of the thermostatic period, the thermostatic device 30 ends the control of the temperature and humidity. If incubating is not included, the time of the immersion treatment is managed. The immersion period is preferably set to a value empirically determined as a time length for sufficiently reducing adhesion. After the elapse of the immersion period, the process advances to step S5. If incubating is not performed in step S4, the thermostatic device 30 may not be included in the cell cryopreservation system 1.

If step S4 is performed, the detachment liquid is removed (step S5). In step S5, the control apparatus 15 operates the waste liquid pump 117 via the driving device 119. The waste liquid pump 117 sucks the detachment liquid from the culture container 40 via the suction pipe 118. Impurities such as the detachment liquid remaining without being sucked exist in the culture container 40.

If step S5 is performed, the iPS cells are cleaned by the cleaning liquid (step S6). In step S6, the control apparatus 15 operates the cleaning liquid pump 113 via the driving device 119. The cleaning liquid pump 113 discharges the cleaning liquid into the culture container 40 via the liquid feed pipe 114. The impurities adhered to the culture container 40 or the iPS cells are washed out by the discharged cleaning liquid. After that, the waste liquid pump 117 sucks the cleaning liquid containing the washed-out impurities from the culture container 40 via the suction pipe 118.

If step S6 is performed, the cryopreservation liquid is discharged to the culture surface 41 of the culture container 40 (step S7). In step S7, the control apparatus 15 operates the cryopreservation liquid pump 115 via the driving device 119. The cryopreservation liquid pump 115 sprays the cryopreservation liquid to the culture surface 41 via the liquid feed pipe 116 and the nozzle 123. Since the adhesion between the iPS cells and the culture surface 41 is weakened by the detachment liquid, the iPS cells are detached from the culture surface 41 by the dynamic action of the droplets of the sprayed cryopreservation liquid.

If step S7 is performed, the cryopreservation liquid in which the iPS cells are suspended (cell-suspended preservation liquid) is recovered to the cryopreservation container 133 (step S8). In step S8, the control apparatus 15 operates the recovery pump 131 via the driving device 134. The recovery pump 131 sucks the cell-suspended preservation liquid from the culture container 40 via the suction branch pipe 141. The sucked cell-suspended preservation liquid is fed to the cryopreservation container 133 via the liquid feed branch pipe 142.

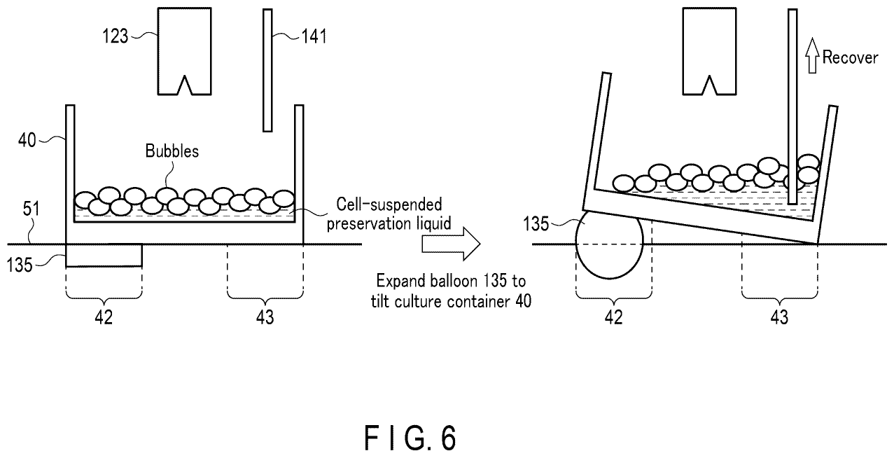

Details of the recovery step (S8) will be described here with reference to FIG. 6. FIG. 6 is a view schematically showing the processing procedure of the recovery step (S8). As shown in the left view of FIG. 6, the cryopreservation liquid is sprayed, thereby generating bubbles of the cryopreservation liquid in the culture container 40. The bubbles tend to be generated on the entire culture surface 41 of the culture container 40. If the iPS cells are stored in the cryopreservation container 133 together with the bubbles, the iPS cells may be damaged by the bubbles breaking. To reduce the risk, the culture container 40 is tilted by the balloon 135, thereby sucking the cell-suspended preservation liquid while avoiding the bubbles.

More specifically, as shown in the right view of FIG. 6, the air compressor 136 expands the balloon 135, thereby raising the rising-side end portion 42 of the culture container 40 and thus tilting the culture container 40. If the culture container 40 is tilted, the cell-suspended preservation liquid gathers to the fixed-side end portion 43. As a result, the depth of the cell-suspended preservation liquid is deeper at the fixed-side end portion 43 than at the rising-side end portion 42. The bubbles are lightweight and therefore form an upper layer. The suction branch pipe 141 is lowered at the fixed-side end portion 43, the distal end portion of the suction branch pipe 141 is made to reach a position deeper than the bubbles, and the cell-suspended preservation liquid is sucked while avoiding the bubbles. If the cell-suspended preservation liquid is sucked while avoiding the bubbles, about 95% of the cell-suspended preservation liquid sprayed to the culture surface 41 is expected to be recovered.

If step S8 is performed, the concentration of the cell-suspended preservation liquid is adjusted (step S9). More specifically, the cryopreservation liquid is added to the cryopreservation container 133 such that the concentration of the cell-suspended preservation liquid in the cryopreservation container 133 obtains a predetermined value. As one example, the predetermined value is a ratio at which the number of iPS cells contained in 200 μl cell-suspended preservation liquid is 3.0×10⁵ or more and 7.0×10⁵ or less. The concentration of the cell-suspended preservation liquid is preferably measured using a flow cytometer, a cell counter, a turbidimetry method, or another arbitrary method. The cryopreservation liquid may be injected, by the cryopreservation liquid pump 115, into the cryopreservation container 133 via the liquid feed pipe 116, or may be injected manually.

If step S9 is performed, the cryopreservation container 133 is stored in the ultra low temperature freezer 21, and the cell-suspended preservation liquid is frozen (step S10). More specifically, the cryopreservation container 133 is separated from the liquid feed branch pipe 142 by an operator or the like and stored in the ultra low temperature freezer 21. The ultra low temperature freezer 21 maintains the inside temperature at about −80° C. within the first temperature range. The iPS cells can thus be frozen at about −80° C. In the ultra low temperature freezer 21, the iPS cells are preserved only for a predetermined period (to be referred to as a first preservation period hereinafter) that is relatively short.

If the iPS cells kept at room temperature are abruptly frozen in the second temperature range which is lower than the first temperature range and in which metabolization is impossible, the iPS cells may be damaged even if the cryopreservation liquid contains a cryoprotectant agent. To reduce damages to the iPS cells caused by the abrupt temperature lowering, freezing in the first temperature range in which metabolization is possible is temporarily performed. The first preservation period is assumed to be a relatively short period, for example, one day. Note that since the iPS cells can be metabolized in the first temperature range, as described above, the upper limit of the first preservation period is one month.

If step S10 is performed, the cryopreservation container 133 is stored in the freezing storage container 22, and the cell-suspended preservation liquid is frozen and stored (step S11). As one example, a liquid nitrogen storage container that is a heat insulating container filled with liquid nitrogen is used as the freezing storage container 22. In this case, the inside of the freezing storage container 22 is maintained at about −180° C. in the second temperature range. Since the iPS cells are frozen in the first temperature range and then frozen in the second temperature range, as described above, damages to the iPS cells caused by freezing at a very low temperature in the second temperature range can be reduced. In the freezing storage container 22, the iPS cells are preserved only for a predetermined period (to be referred to as a second preservation period hereinafter) that is relatively long. In the second temperature range, the iPS cells cannot be metabolized and can therefore be preserved for several years.

Thus, detachment/cryopreservation by the cell cryopreservation system 1 ends.

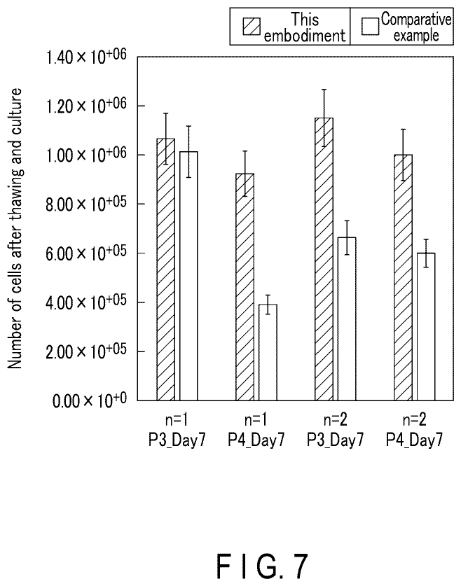

FIG. 7 is a view showing comparison of the number of iPS cells after thawing and culture between this embodiment and a comparison example. In “this embodiment”, the cryopreservation liquid was sprayed to detach cells, and the cell-suspended preservation liquid was frozen without performing liquid replacement. In the comparative example, detachment was performed by a technique using a saline solution, liquid replacement using centrifugal separation was executed for the saline solution in which iPS cells were suspended, the saline solution was replaced with the cryopreservation liquid, and freezing was performed. The ordinate of FIG. 7 indicates the number of iPS cells after thawing and culture. The abscissa of FIG. 7 indicates classifications of culture conditions. “n=1, P3_Day7” indicates that iPS cells of cell type label “1” were cultured under conditions of passage number “3” and culture period “7 days”. Similarly, “n=1, P4 Day7” indicates that iPS cells of cell type label “1” were cultured under conditions of passage number “4” and culture period “7 days”, “n=2, P3 Day7” indicates that iPS cells of cell type label “2” were cultured under conditions of passage number “3” and culture period “7 days”, and “n=2, P4 Day7” indicates that iPS cells of cell type label “2” were cultured under conditions of passage number “4” and culture period “7 days”.

As shown in FIG. 7, the number of iPS cells is larger in this embodiment than in the comparative example under any culture conditions. It can be considered that one of the factors is that the liquid replacement step is unnecessary in this embodiment, unlike the comparative example, and damages to the iPS cells are reduced. Hence, it can be found that this embodiment is superior to the comparative example from the viewpoint of the number of iPS cells after thawing.

The processing procedure of detachment/cryopreservation shown in FIG. 4 is merely an example, and various elements can be removed, added and/or changed without departing the scope of the present invention.

As one example, if the concentration of the cell-suspended preservation liquid is already suitable, or if the concentration need not be adjusted, the adjustment step (S9) can be omitted. As another example, if the cell-suspended preservation liquid need not be frozen, the freezing step (S10) and the cryopreservation step (S11) can be omitted. As still another example, if the cryopreservation liquid can be sprayed without adding the detachment liquid, the addition step (S3), the incubating step (S4), the removal step (S5), and the cleaning step (S6) can be omitted.

As another example, the mechanism 136 that tilts the culture container 40 is not limited to the method of expanding a balloon. As one example, the tilting mechanism 135 can also be implemented by a mechanism that tilts the culture container 40 by extending a cylinder and pushing the rising-side end portion 42 of the culture container 40 upward or a mechanism that tilts the culture container 40 by hanging up the rising-side end portion 42 using a wire or the like.

As another example, detachment of iPS cells is not limited to the method of spraying the cryopreservation liquid. As one example, the cryopreservation liquid may be discharged, by the cryopreservation liquid pump 115, to the iPS cells via the liquid feed pipe 116, and the iPS cells may be detached from the culture surface 41 by the dynamic action of the discharged cryopreservation liquid. As another example, the cryopreservation liquid may be fed, by the cryopreservation liquid pump 115, into the culture container 40 via the liquid feed pipe 116, and the iPS cells may be detached from the culture surface 41 by a water stream generated by the liquid feed. As still another example, the cryopreservation liquid may be fed, by the cryopreservation liquid pump 115, into the culture container 40 via the liquid feed pipe 116, the culture container 40 may be vibrated by the tilting mechanism 135, and the iPS cells may be detached from the culture surface 41 by a water stream generated by the vibration.

As another example, the tilting mechanism 135 can also be used at the time of suction of various kinds of waste liquids by the waste liquid pump 117. More specifically, when the waste liquid pump 117 is going to suck various kinds of waste liquids via the suction pipe 118, the tilting mechanism 135 tilts the culture container 40 to increase the depth of the waste liquid at a position where the suction pipe 118 is inserted. Thus, various kinds of waste liquids can easily be collected from the culture container 40.

As still another example, all steps S1 to S8 need not be executed by the detachment mechanism 11, and some or all of steps S1 to S8 may be manually executed by an operator. As one example, as for the discharge step (S7), the operator may discharge the cryopreservation liquid to the culture surface 41 using a pipette or the like, thereby detaching the iPS cells from the culture surface 41. As for the recovery step (S8), the operator may suck the cell-suspended preservation liquid from the culture container 40 using a pipette or the like and discharge the sucked cell-suspended preservation liquid to the cryopreservation container 133, thereby recovering the cell-suspended preservation liquid.

As described above, the cell detachment method according to this embodiment includes a discharge step and a recovery step. In the discharge step, a cryopreservation liquid for cells is discharged toward a culture surface of a culture container in contact with iPS cells, where the iPS cells are cultured, thereby detaching the iPS cells from the culture surface. In the recovery step, the detached iPS cells are recovered together with the discharged cryopreservation liquid.

According to the above-described configuration, it is possible to recover the cryopreservation liquid in which the cells are suspended (cell-suspended preservation liquid) from the culture container. In other words, since it is possible to directly freeze or cryopreserve the recovered cell-suspended preservation liquid, the liquid replacement step can be omitted, unlike a case where cells are detached from the culture surface using a solution for detachment such as a saline solution. Hence, in this embodiment, the labor required for the liquid replacement step can be reduced. In the liquid replacement step, many of the cells suspended in the cell-suspended detachment liquid cannot be recovered, and loss of cells is large. However, in this embodiment, since the cell-suspended preservation liquid recovered from the culture container can directly be frozen, loss of cells associated with the liquid replacement step can be eliminated. Also, in this embodiment, since the liquid replacement step is unnecessary, the cells recovered from the culture container can quickly be frozen, and damages to the cells associated with exposure to room temperature can be reduced as compared to a case where the liquid replacement step is executed.

According to at least one embodiment described above, it is possible to recover cells from the culture container while reducing loss of cells.

Note that the term “processor” used in the above explanation means, for example, a CPU, a GPU, or a circuit such as an Application Specific Integrated Circuit (ASIC) or a programmable logic device (for example, a Simple Programmable Logic Device (SPLD), a Complex Programmable Logic Device (CPLD), or a Field Programmable Gate Array (FPGA)). The processor implements a function by reading out a program stored in a storage circuit and executing it. Note that the program may directly be incorporated in the circuit of the processor, instead of storing the program in the storage circuit. In this case, the processor implements a function by reading out the program incorporated in the circuit and executing it. On the other hand, if the processor is, for example, an ASIC, the function is directly incorporated as a logic circuit in the circuit of the processor, instead of storing the program in the storage circuit. Note that the processor according to this embodiment is not necessarily configured as a single circuit for each processor, and a plurality of independent circuits may be combined to form one processor and implement the function. Furthermore, a plurality of constituent elements in FIGS. 1 and 2 may be integrated into one processor to implement the function.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cell detachment method comprising:
a detachment step of discharging a liquid used for cryopreservation of cells toward a surface of a culture container in contact with the cells, where the cells are cultured, thereby detaching the cells from the surface; and
a recovery step of recovering the detached cells together with the discharged liquid used for cryopreservation.

2. The method according to claim 1, wherein the liquid used for cryopreservation is a solution containing a cryoprotectant agent and a culture medium.

3. The method according to claim 1, wherein the liquid used for cryopreservation contains a cryoprotectant agent, and
the cryoprotectant agent contains dimethyl sulfoxide, glycerol, polyethylene glycol, propylene glycol, glycerin, polyvinyl pyrrolidone, sorbitol, dextran and/or trehalose.

4. The method according to claim 1, wherein in the recovery step, the detached cells are recovered together with the discharged liquid used for cryopreservation while avoiding bubbles generated by the discharge.

5. The method according to claim 1, wherein in the detachment step, the cells are detached from the surface by discharging the liquid used for cryopreservation as a plurality of droplets to the surface, discharging the liquid used for cryopreservation to the cells, or a water stream generated by feed or a vibration of the liquid used for cryopreservation.

6. The method according to claim 1, further comprising, at a preceding stage of the detachment step, an addition step of adding a detachment liquid to the cells included in the culture container to weaken adhesion of the cells to the surface.

7. The method according to claim 6, wherein the detachment liquid contains a proteolytic enzyme and/or a chelating agent.

8. The method according to claim 1, further comprising, at a preceding stage of the detachment step, a cleaning step of cleaning, using a cleaning liquid, the culture container in which the cells are adhered to the surface, thereby removing an impurity from the culture container.

9. The method according to claim 8, wherein the cleaning liquid is one of a saline solution and a liquid culture medium.

10. The method according to claim 1, further comprising an adding step of, to adjust a concentration of a suspension of the recovered cells and the liquid used for cryopreservation to a predetermined value, adding the liquid used for cryopreservation to the suspension.

11. The method according to claim 1, further comprising a freezing step of freezing a suspension formed by suspending the recovered cells in the liquid used for cryoproservation.

12. The method according to claim 11, wherein
the freezing step includes:
a first freezing step of freezing the suspension in a first temperature range in which the cells can be metabolized; and
a second freezing step of, after the first freezing step, freezing and preserving the suspension in a second temperature range which is lower than the first temperature range and in which the cells cannot be metabolized.

13. A cell detachment method comprising:
a detachment step of discharging a liquid toward a surface of a culture container in contact with the cells, where the cells are cultured, thereby detaching the cells from the surface;
a recovery step of recovering the detached cells together with the discharged liquid; and
a freezing step of freezing the recovered cells and the liquid.

14. A cell detachment apparatus comprising:
a detachment mechanism configured to discharge a liquid used for cryopreservation of cells toward a surface of a culture container in contact with the cells, where the cells are cultured, thereby detaching the cells from the surface; and
a recovery mechanism configured to recover the detached cells together with the discharged liquid used for cryopreservation.

15. The apparatus according to claim 14, wherein
the recovery mechanism includes:
a suction pipe in which the liquid used for cryopreservation is distributed;
a cryopreservation container detachably provided on the suction pipe; and
a pump configured to suck, via the suction pipe, a suspension formed by suspending the detached cells in the discharged liquid used for cryopreservation and feeding the suspension to the cryopreservation container.

16. The apparatus according to claim 15, further comprising a cryopreservation unit including a freezer configured to store the cryopreservation container and freeze the suspension included in the cryopreservation container.

17. The apparatus according to claim 15, wherein
the recovery mechanism further comprises a tilting mechanism configured to tilt the culture container with respect to a horizontal direction such that a depth of the suspension at a first position of the culture container, where the suction pipe is inserted, is deeper than the depth of the suspension at another second position, and
the pump sucks the suspension via the suction pipe inserted to the first position.

18. The apparatus according to claim 14, wherein the detachment mechanism includes a nozzle configured to spray the liquid used for cryopreservation to the surface.

19. A cell cryopreservation system comprising:
a detachment mechanism configured to discharge a liquid toward a surface of a culture container in contact with the cells, where the cells are cultured, thereby detaching the cells from the surface;
a recovery mechanism configured to recover the detached cells together with the discharged liquid; and
a cryopreservation unit configured to freeze the recovered cells and the liquid.
